(19) United States
(12) Patent Application Publication
(10) Pub. No.: US 2025/0289741 A1
(43) Pub. Date: Sep. 18, 2025

(54) ASPERGILLUS SP. DH4 WITH AEROBIC DENITRIFICATION ENHANCED BY INORGANIC ELECTRON DONOR AND USE THEREOF

(71) Applicant: Xi'an University of Architecture and Technology, Xi 'an (CN)

(72) Inventors: Haihan Zhang, Xi 'an (CN); Ben Ma, Xi 'an (CN); Sixuan Pan, Xi 'an (CN); Fengrui Li, Xi 'an (CN)

(21) Appl. No.: 19/069,227

(22) Filed: Mar. 4, 2025

(30) Foreign Application Priority Data
Mar. 14, 2024 (CN) 202410288115.9

(51) Int. Cl. C02F 3/34; C02F 3/02; C02F 101/16; C02F 103/00; C12N 1/14; C12R 1/66

(52) U.S. Cl. CPC C02F 3/346; C02F 3/02; C02F 3/348; C12N 1/145; C02F 2101/163; C02F 2103/007; C12R 2001/66

(57) ABSTRACT

An Aspergillus sp. DH4 with aerobic denitrification enhanced by an inorganic electron donor and its application are provided, which relates to the field of microbial technologies. The Aspergillus sp. DH4 is preserved at China Center for Type Culture Collection (CCTCC), a preservation address is Wuhan University, 299 Bayi Road, Wuchang District, Wuhan City, China, a preservation number is CCTCC NO: M20232690, and a preservation date is Dec. 27, 2023.

### CROSS-REFERENCE TO RELATED APPLICATION

This application claims priority to Chinese Patent Application No. 202410288115.9, filed Mar. 14, 2024, which is herein incorporated by reference in its entirety.

### TECHNICAL FIELD

The disclosure relates to the field of microbial technologies, and more particularly to an Aspergillus sp. DH4 with aerobic denitrification enhanced by an inorganic electron donor and its use.

### STATEMENT REGARDING SEQUENCE LISTING

The sequence listing associated with this application is provided in text format in lieu of a paper copy and is hereby incorporated by reference into the specification. The name of the XML file containing the sequence listing is 25002MYZ-USP1-SL.xml. The XML file is 2,513 bytes; is created on Feb. 25, 2025; and is being submitted electronically via patent center.

### BACKGROUND

Biological nitrogen removal refers to a process of converting, by microorganisms, nitrogen-containing compounds in water into dinitrogen oxide or nitrogen gas through biochemical actions. To date, the biological nitrogen removal has been widely applied in the treatment of nitrogen-polluted water bodies, such as wastewater treatment plants, polluted waterways, source reservoirs, and constructed wetlands. The cost-effectiveness, high efficiency, and environmental friendliness of the biological nitrogen removal have made it a primary mode for nitrogen pollution control in water bodies.

Currently, research on the mechanisms of aerobic denitrification mainly focuses on the theories of co-respiration and denitrification enzyme systems. The co-respiration theory suggests that the redox potential of nitrate is close to that of oxygen (O₂). Therefore, an aerobic denitrifying strain can perform nitrate respiration (i.e., aerobic denitrification) concurrently with aerobic respiration. Compared to traditional anaerobic denitrification, the aerobic denitrification can simultaneously carry out nitrification and denitrification processes under the same dissolved oxygen conditions. The aerobic denitrifying strain is a key microorganism in the processes of simultaneous nitrification and denitrification (SND) and partial nitrification-denitrification.

Compared to the traditional anaerobic denitrification, the aerobic denitrification can simultaneously carry out the nitrification and denitrification processes under the same dissolved oxygen conditions. With the development of society and the increasing environmental awareness of humans, the carbon to nitrogen (C/N) ratio in wastewater has decreased, leading to a lack of carbon sources for traditional heterotrophic denitrification. Therefore, mixotrophic denitrification is a new alternative. The mixotrophic denitrification refers to the simultaneous presence of a reduced inorganic electron donor and organic matter in a reaction system, both of which can serve as an electron donor for microbial denitrification. However, there are relatively few reports on the mixotrophic denitrification in the related art.

### SUMMARY

Removing nitrogen from water bodies plays a crucial role in controlling eutrophication. Among nitrogen removal technologies for the water bodies, biological nitrogen removal is a highly efficient and cost-effective method.

In order to address deficiencies in the related art, and in response to the above problems, the disclosure aims at providing an Aspergillus sp. DH4 with denitrification performance enhanced by iron as an inorganic electron donor.

The Aspergillus sp. DH4 with aerobic denitrification enhanced by the inorganic electron donor, is preserved at China Center for Type Culture Collection (CCTCC), a preservation address is Wuhan University, 299 Bayi Road, Wuchang District, Wuhan City, China, a preservation number is CCTCC NO: M20232690 and a preservation date is Dec. 27, 2023.

The Aspergillus sp. DH4 is applied in restoring a nitrogen-containing water body. For example, the Aspergillus sp. DH4 is added to the nitrogen-containing water body.

In an embodiment, a carbon to nitrogen (C/N) ratio of the nitrogen-containing water body is in a range of 1-2.5, and a temperature of the nitrogen-containing water body is in a range of 25-30 degrees Celsius (° C.). A rotation speed of the nitrogen-containing water body is in a range of 80-120 revolutions per minute (rpm).

In an embodiment, during the restoring a nitrogen-containing water body, a usage amount of the Aspergillus sp. DH4 is 1%-5% of the nitrogen-containing water body in percentages by weight.

In an embodiment, the inorganic electron donor is zero-valent iron.

In an embodiment, the zero-valent iron is a zero-valent iron powder or a zero-valent iron rod.

A method for removing nitrate from a polluted water body includes: inoculating the Aspergillus sp. DH4 into the nitrogen-containing water body, where the zero-valent iron is added as the inorganic electron donor.

Iron, as the fourth most abundant element in the earth crust, plays a significant role in the biogeochemical cycling of the nitrogen in ecosystems. Utilizing reduced iron as an electron donor for denitrification, the coupling of iron oxidation and nitrogen reduction holds broad application prospects in nitrogen removal from water sources. The addition of the zero-valent iron may involve both chemical and biological denitrification processes. During chemical denitrification, the oxidation of the zero-valent iron releases electrons that serve as electron donors for nitrate reduction. In biological denitrification, the generated ferrous ion (Fe²⁺) may further participate in Fe²⁺-mediated autotrophic denitrification.

Without adding exogenous inorganic electron donors, the Aspergillus sp. DH4 has a nitrate removal rate of approximately 30% in a medium with a C/N ratio of 2. The accumulation and subsequent removal of nitrite are observed between days 6 and 10, indicating the occurrence of aerobic denitrification.

The effect of different iron added dosages on the nitrogen removal performance of the Aspergillus sp. DH4 is investigated when adding the iron as the inorganic electron donor. To investigate the effect of different iron added dosages on the nitrogen removal performance of the Aspergillus sp. DH4, the iron is added at concentrations of 5, 10, 15, and 20 grams per liter (g/L). The removal of nitrate nitrogen (NO₃⁻—N), ammonia nitrogen (NH₄⁺—N), nitrite nitrogen (NO₂⁻—N), and total nitrogen (TN) in the medium with the C/N ratio of 2 by the Aspergillus sp. DH4 under different iron added dosages is detected every 2 days.

As the added dosage of iron increases, the nitrate removal rate of the Aspergillus sp. DH4 gradually accelerates, with complete nitrate removal achievable within 6 days at a highest added dosage. However, the accumulation of the NH₄⁺—N also increases continuously, indicating that the proportion dominated by the chemical action of the iron is gradually increasing.

The optimal added dosage of the iron as the inorganic electron donor for the denitrification of the Aspergillus sp. DH4 is determined. Based on the analysis of experimental results, the iron added dosage of 10 g/L is selected as the optimal added dosage, which achieves a relatively fast nitrate removal rate with minimal accumulation of the NH₄⁺—N, which could ultimately be removed through nitrification.

Compared to the related art, the disclosure has the following beneficial effects.

The disclosure obtains the Aspergillus sp. DH4 with the aerobic denitrification performance enhanced by the iron as the inorganic electron donor. The Aspergillus sp. DH4 with the aerobic denitrification enhanced by the inorganic electron donor is preserved at CCTCC, a preservation address is Wuhan University, 299 Bayi Road, Wuchang District, Wuhan City, China, a preservation number is CCTCC NO: M20232690 and a preservation date is Dec. 27, 2023. When performing aerobic denitrification not enhanced by the iron as the inorganic electron donor, the Aspergillus sp. DH4 achieves a nitrate removal rate of approximately 30% in a medium with a C/N ratio of 2. The addition of 20 g/L iron allows for complete nitrate removal by the sixth day. When 10 g/L of the iron is added, the nitrate removal rate is faster, and there is less accumulation of the NH₄⁺—N, making it the optimal added dosage. Valuable reference for the denitrification performance of aerobic denitrifying strains enhanced by inorganic electron donors is provided.

### BRIEF DESCRIPTION OF DRAWINGS

FIG. 1A and FIG. 1B respectively illustrate a phylogenetic tree and strain morphology of an Aspergillus sp. DH4 of the disclosure.

FIGS. 2A-2D respectively illustrate schematic diagrams of nitrogen removal performance of the Aspergillus sp. DH4 of the disclosure at different rotation speeds; where FIG. 2A corresponds to a case at 40 rpm; FIG. 2B corresponds to a case at 80 rpm; FIG. 2C corresponds to a case at 120 rpm; and FIG. 2D corresponds to a case at 160 rpm.

FIGS. 3A-3C respectively illustrate schematic diagrams of the nitrogen removal performance of the Aspergillus sp. DH4 of the disclosure at different C/N ratios; where FIG. 3A corresponds to a case at C/N=1; FIG. 3B corresponds to a case at C/N=1.5; and FIG. 3C corresponds to a case at C/N=2.5.

FIGS. 4A-4D respectively illustrate schematic diagrams of the nitrogen removal performance of the Aspergillus sp. DH4 of the disclosure at different iron added dosages; where FIG. 4A represents concentration changes of NO₃⁻—N; FIG. 4B represents concentration changes of NO₂⁻—N; FIG. 4C represents concentration changes of NH₄⁺—N; and FIG. 4D represents concentration changes of TN.

FIGS. 5A-5D respectively illustrate schematic diagrams of the nitrogen removal performance of the Aspergillus sp. DH4 of the disclosure at different temperatures; where FIG. 5A corresponds to a case at a temperature of 5° C.; FIG. 5B corresponds to a case at a temperature of 10° C.; FIG. 5C corresponds to a case at a temperature of 15° C.; and FIG. 5D corresponds to a case at a temperature of 20° C.

FIGS. 6A-6I respectively illustrate zero-order, half-order, and first-order kinetic models of the Aspergillus sp. DH4 at different C/N ratios, different rotation speeds, and different temperatures; where FIGS. 6A-6C respectively represent the zero-order, half-order, and first-order kinetic models at different C/N ratios respectively, FIGS. 6D-6F respectively represent the zero-order, half-order, and first-order kinetic models at different rotation speeds respectively, and FIGS. 6G-6I respectively represent the zero-order, half-order, and first-order kinetic models at different temperatures respectively.

FIG. 7A and FIG. 7B respectively illustrate nitrogen removal performance, and cell growth and dissolved organic carbon (DOC) removal of the Aspergillus sp. DH4 of the disclosure under optimal conditions.

FIG. 8 illustrates release of ferric ions (Fe³) and Fe²⁺ in a system of the Aspergillus sp. DH4 of the disclosure.

FIG. 9 illustrates kinetic models for nitrate removal under different parameters for the Aspergillus sp. DH4 of the disclosure.

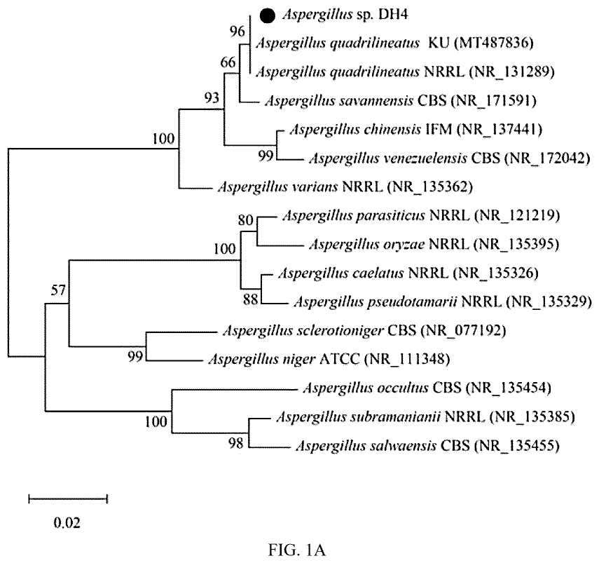

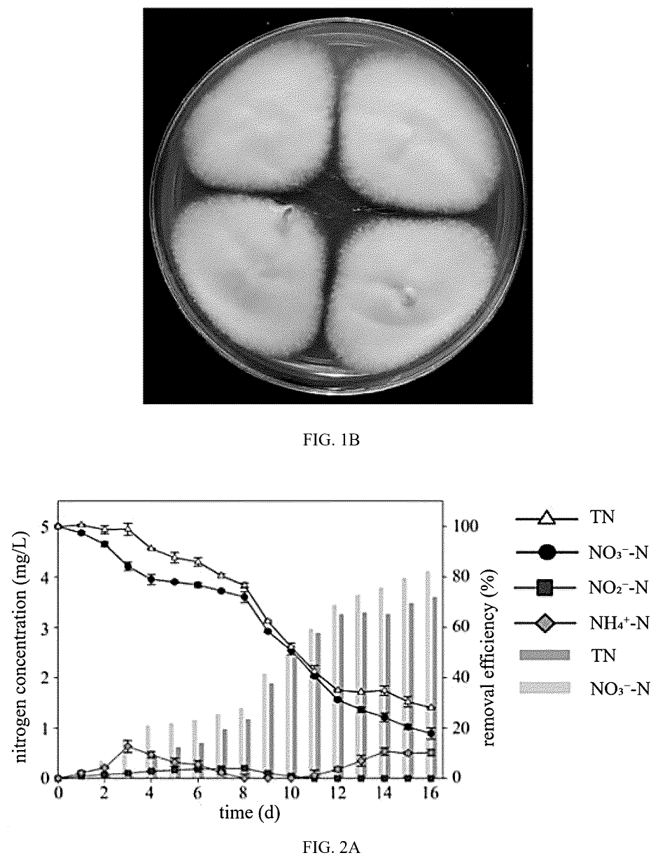

### DETAILED DESCRIPTION OF EMBODIMENTS

The embodiments of the disclosure are described in detail below, but it should be understood that the scope of protection of the disclosure is not limited by the illustrated embodiments. Based on the embodiments of the disclosure, all other embodiments obtained by those skilled in the art without creative labor are within the scope of protection of the disclosure. The experimental methods described in each embodiment of the disclosure are conventional methods unless otherwise specified.

A rose Bengal agar medium includes a formulation consisting of: 5 g/L of peptone, 0.025 g/L of C₂₀H₂Cl₄I₄Na₂O₅ (rose Bengal), 10 g/L of C₆H₁₂O₆ (glucose), 0.002 g/L of C₆H₄Cl₂N₂O₂ (4,5-dichloro-2-nitroaniline), 1 g/L of KH₂PO₄ (potassium dihydrogen phosphate), 0.1 g/L of C₁₁H₁₂Cl₂N₂O₅ (chloramphenicol), and 0.5 g/L of MgSO₄ (magnesium sulfate), with a pH in a range of 5.6-5.8; and 20 g/L of agar powder is added to the formulation to prepare a fungal solid medium.

A denitrification liquid medium (DM) uses a conventional denitrification liquid medium known in the related art, with a formulation and a preparation method including: 0.108 g/L of KNO₃ (potassium nitrate), 1.5 g/L of KH₂PO₄, 0.413 g/L of glucose, 0.1 g/L of MgSO₄·7H₂O (magnesium sulfate heptahydrate), 5.0 g/L of Na₂HPO₄·12H₂O (disodium hydrogen phosphate dodecahydrate), and 2 milliliters (mL) of trace element stock solution are added to ultrapure water followed by adjusting a volume to 1 liter (L) and stirring until completely dissolved to obtain first mixed solution, a pH of the first mixed solution is adjusted to 7.0-7.2, and then the first mixed solution is sterilized at 121° C. for 30 minutes (min) and stored for later use.

A formulation and a preparation method of the trace element stock solution are as follows. 4.4 milligrams (mg) of ZnSO₄ (zinc sulfate), 100 mg of ethylene diamine tetraacetic acid (EDTA), 10.2 mg of MnCl₂·4H₂O (manganese(II) chloride tetrahydrate), 11 mg of CaCl₂) (calcium chloride), 10 mg of FeSO₄·7H₂O (ferrous sulfate heptahydrate), 3.2 mg of CuSO₄·5H₂O (copper(II) sulfate pentahydrate), 2.2 mg of (NH₄)₆Mo₇O₂₄·4H₂O (ammonium molybdate tetrahydrate), and 3.2 mg of CoCl₂·6H₂O (cobalt(II) chloride hexahydrate) are added to ultrapure water followed by adjusting a volume to 1 L, and stirring until completely dissolved to obtain second mixed solution, a pH of the second mixed solution is adjusted to 7.0-7.2, and then the second mixed solution is sterilized at 121° C. for 30 min and stored for later use.

In the disclosure, the term “iron” refers to zero-valent iron, which can be in a form of an iron powder or an iron rod. The unit “g/L” for iron added dosage indicates grams of the iron added per liter of treated water.

In a fungal suspension of an Aspergillus sp. DH4 with a weight concentration of 1%, there are approximately 5×10⁵ cells.

### Embodiment 1

Strain isolation and purification are as follows.

Denitrifying fungal strains are enriched and isolated from overlying water samples of a water source reservoir.

Fresh sediment and floating water samples are collected from Xili Reservoir in Shenzhen, China, located at 113° 57′6″ east (E), and 22° 35′37″ north (N), with a sampling depth of 0-0.5 meters (m). In this experiment, the water and sediment are placed in a beaker of 2 L followed by continuous aerating to maintain a dissolved oxygen concentration of 6.6 milligrams per liter (mg/L). After one month of continuous aerobic acclimatization, supernatant of the water sample in the beaker is extracted, and then diluted by using a 10-fold serial dilution method, followed by being coated onto rose Bengal agar mediums to obtain coated plates.

The coated plates are inverted and incubated in a biochemical incubator at 30° C. until visible colonies formed, with each mixed microbial sample coated on three plates. All materials used are sterilized at 121° C. for 30 min and then operated in a sterile clean bench. After colony formation, plate coating is repeated 3-5 times until distinct single colonies are obtained.

A strain obtained from the single colonies is subjected to internal transcribed spacer (ITS) sequencing to obtain its genetic information.

The strain is identified as Aspergillus sp. (a species within the genus Aspergillus), and named DH4. The colonies of the strain appear white and radiating. The phylogenetic tree of the Aspergillus sp. DH4 is constructed by using the neighbor-joining method. Aspergillus quadrilineatus KU and Aspergillus quadrilineatus NRRL show a genetic sequence similarity of over 99% with the Aspergillus sp. DH4, indicating that the strain DH4 belongs to the genus Aspergillus. A length of the ITS gene of the Aspergillus sp. DH4 is 533 base pairs (bp), and the sequence of the Aspergillus sp. DH4 is CTGCGGAAGGATCATTACCGAGTGCGGGCTGCCTCCGGGCGCCCAACCTCCCACC CGTGACTACCTAACACTGTTGCTTCGGCGGGGAGCCCCCTAGGGGCGAGCCGCCG GGGACCACTGAACTTCATGCCTGAGAGTGATGCAGTCTGAGCCTGAATACAAATC AGTCAAAACTTTCAACAATGGATCTCTTGGTTCCGGCATCGATGAAGAACGCAGC GAACTGCGATAAGTAATGTGAATTGCAGAATTCAGTGAATCATCGAGTCTTTGAA CGCACATTGCGCCCCCTGGCATTCCGGGGGGCATGCCTGTCCGAGCGTCATTGCT GCCCTCAAGCCCGGCTTGTGTGTTGGGTCGTCGTCCCCCCCGGGGGACGGGCCCG AAAGGCAGCGGCGGCACCGTGTCCGGTCCTCGAGCGTATGGGGCTTTGTCACCCG CTCGATTAGGGCCGGCCGGGCGCCAGCCGGCGTCTCCAACCTTATTTTTCTCAGG TTGACCTCGGATCAGGTAGGGATACCCGCTGAACTTAA, denoted as SEQ ID NO: 1.

### Embodiment 2

Nitrogen removal performance of the Aspergillus sp. DH4 with an inorganic electron donor added at different rotation speeds is tested.

The Aspergillus sp. DH4 is inoculated into a sterilized DM medium with 5 mg/L of nitrogen and a C/N ratio of 2, and 10 g/L of iron is added as the inorganic electron donor. Then, different rotation speeds of 40, 80, 120, and 160 rpm are set as comparative experiments, and the sterilized DM medium with the Aspergillus sp. DH4 inoculated are placed at 30° C. in a biochemical shaker incubator for culturing. Samples are taken every 2 days and filtered through pre-combusted 0.45 micrometers (μm) glass fiber filters (GF/F), and concentrations of NO₃⁻—N, NH₄⁺—N, NO₂⁻—N, and TN are measured under different rotation speeds.

The rotation speed reflects the dissolved oxygen (DO) concentration, and changes of the DO concentration is crucial for the Aspergillus sp. DH4 as an aerobic denitrifying strain.

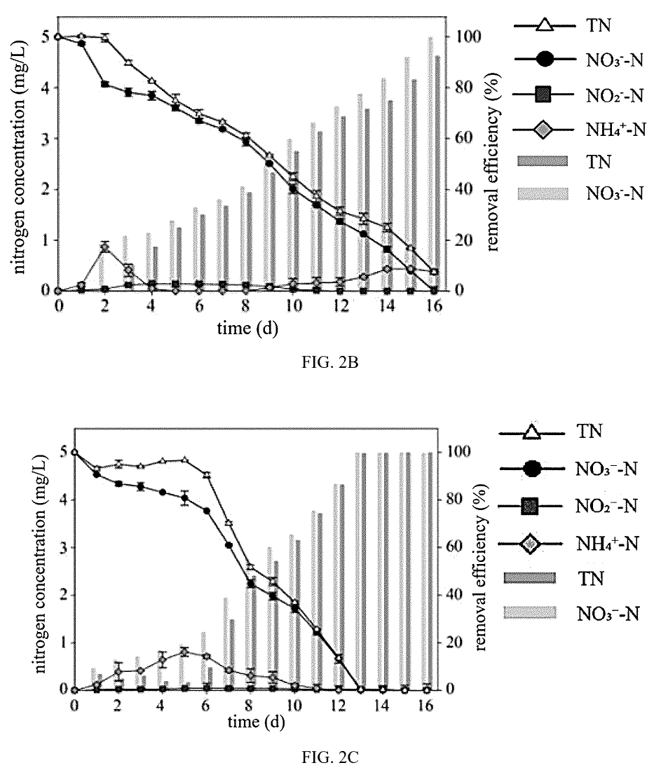

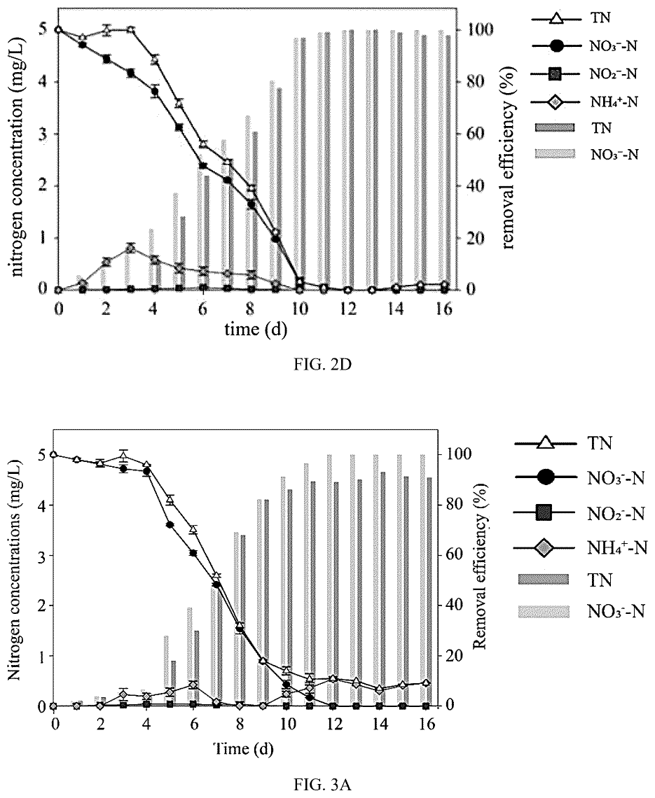

FIGS. 2A-2D show that when the rotation speed is 40 rpm, the nitrate removal rate of the Aspergillus sp. DH4 decreases to 82.20%, due to insufficient DO available for the aerobic denitrifying strain, which is unfavorable for denitrification. Meanwhile, the insufficient DO may lead to a reduced oxidation rate of the iron, a decreased supply of electron donors, and consequently, a lower denitrification rate. Moreover, there is some accumulation of NH₄⁺—N in the later stages of cultivation, reaching up to 0.53 mg/L, which might be caused by the autolysis of dead microbial cells.

FIGS. 2A-2D also indicate that as the rotation speed increases, the nitrogen removal rate of the Aspergillus sp. DH4 continuously rises, with the time required to achieve 100% nitrate removal shortening from 16 days to 11 days. This could be attributed to the increased DO concentration, which accelerates mass transfer rates of oxygen and nitrate, and the oxidation rate of the iron, thereby enhancing the activity of nitrogen metabolism enzymes. When examining the iron added dosage, at an iron concentration of 10 g/L, a C/N ratio of 2, a rotation speed of 120 rpm, and a temperature of 30° C., the TN removal rate reaches 100%, and no NH₄⁺—N accumulation is observed at rotation speeds of 160 rpm and 120 rpm. Throughout the experiment, some accumulation of nitrite is observed at rotation speeds of 40 rpm and 80 rpm, likely due to insufficient DO supply, which reduces the activity of nitrite reductase.

The disclosure selects a rotation speed of 120 rpm as the optimal condition for the Aspergillus sp. DH4, and further studies are conducted to investigate factors affecting the nitrogen removal performance of the Aspergillus sp. DH4.

### Embodiment 3

Nitrogen removal performance of the Aspergillus sp. DH4 with an inorganic electron donor added at different C/N ratios is tested.

The Aspergillus sp. DH4 with a weight concentration of 1% is inoculated into a sterilized DM medium with 5 mg/L of nitrogen, and 10 g/L of iron is added as the inorganic electron donor; then, the sterilized DM medium with the Aspergillus sp. DH4 inoculated are placed in a biochemical shaker incubator at 30° C. and 130 rpm for culturing for 48 hours (h) until a logarithmic growth phase. Different C/N ratios of 1, 1.5, and 2.5 are set as comparative experiments. Samples are taken every 2 days and filtered through pre-combusted 0.45 μm GF/F, and concentrations of NO₃⁻—N, NH₄⁺—N, NO₂⁻—N, and TN are measured under different C/N ratios.

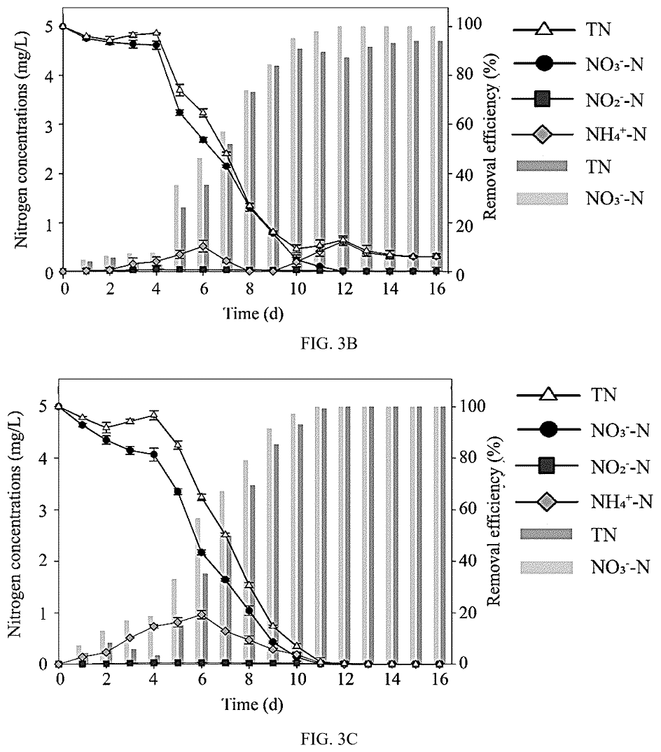

FIGS. 3A-3C show the nitrogen removal rates of the Aspergillus sp. DH4 at the C/N ratios of 1, 1.5, and 2.5, examining the effects of different C/N ratios on nitrogen reduction by the Aspergillus sp. DH4. When the C/N ratio decreases from 2.5 to 1.5 and 1, the TN removal rate of the Aspergillus sp. DH4 decreases from 100% to 90% and 94%, respectively. In each system, the NO₃⁻—N could be completely removed, but there is a re-accumulation of the NH₄⁺—N in the later stages of cultivation. This may be due to the release of the NH₄⁺—N caused by cell death as the cells entered the decline phase, indicating that under low C/N ratios, the limited availability of organic carbon for the Aspergillus sp. DH4 may restrict the nitrogen removal performance and cellular activity of the Aspergillus sp. DH4.

Under low C/N ratios with the addition of the inorganic electron donor, the Aspergillus sp. DH4 could achieve complete TN removal, albeit at different rates. As the C/N ratio increases to 2.5, the TN removal rate of the Aspergillus sp. DH4 reaches complete removal by the eleventh day, and there is no further accumulation of the NH₄⁺—N in the later stages, indicating that increasing the C/N ratio can enhance the TN removal efficiency of the Aspergillus sp. DH4.

Considering the actual engineering application scenarios that water source reservoirs are often oligotrophic, the disclosure selects the lower nutrient condition of C/N=2, which can achieve complete nitrogen removal, for further investigation into the factors affecting the nitrogen removal performance of the Aspergillus sp. DH4.

### Embodiment 4

Nitrogen removal performance of the Aspergillus sp. DH4 added with different concentrations of an inorganic electron donor is tested.

The Aspergillus sp. DH4 with a weight concentration of 1% is inoculated into a sterilized DM medium with 5 mg/L of nitrogen; then, under conditions of a C/N ratio of 2, a temperature of 30° C. and a rotation speed of 120 rpm, different iron added dosages are set as comparative experiments, and concentrations of NO₃⁻—N, NH₄⁺—N, NO₂⁻—N, and TN are measured under different iron added dosages.

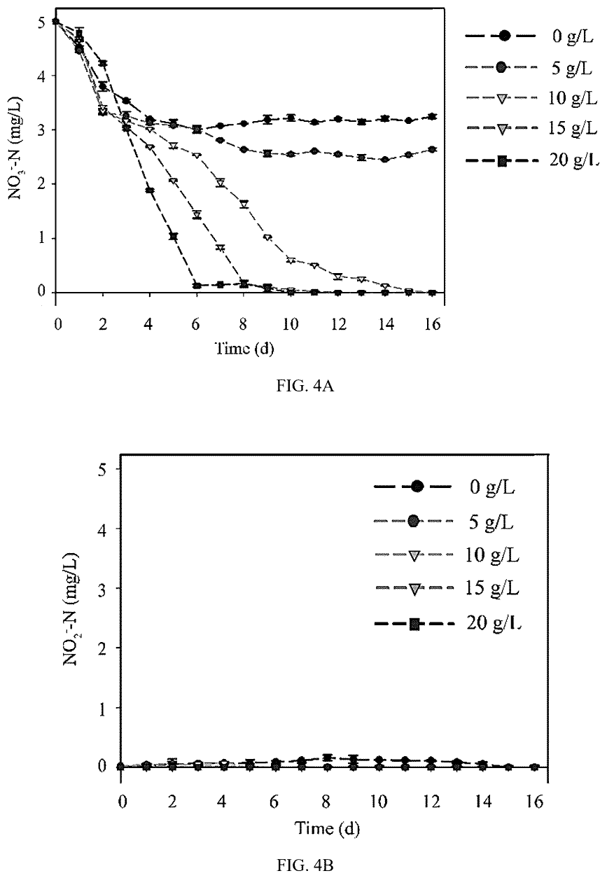

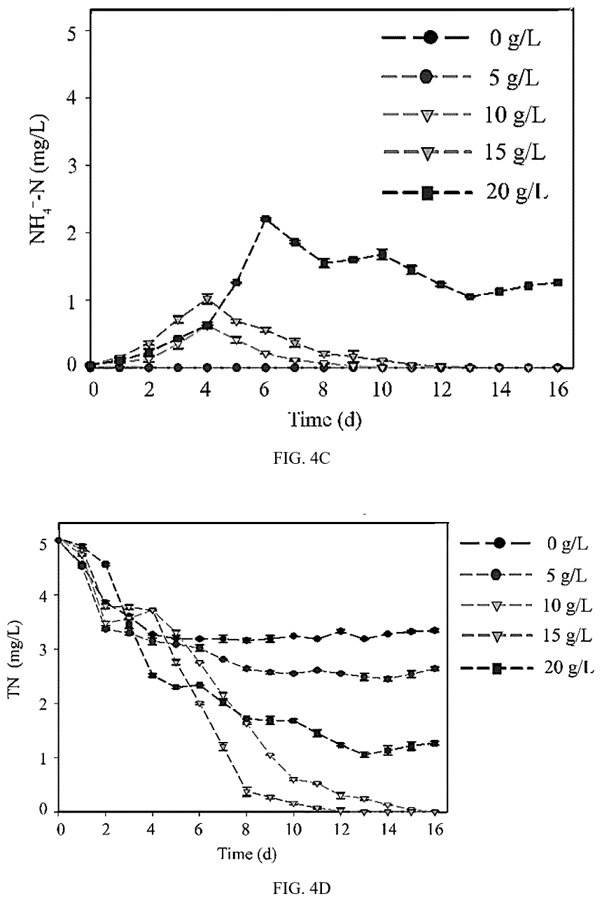

FIGS. 4A-4D show that, without adding iron as the inorganic electron donor, the nitrate removal rate of the Aspergillus sp. DH4 in the medium is around 30%. A noticeable accumulation and subsequent removal of nitrite are observed between the sixth day and the tenth day, indicating the occurrence of aerobic denitrification. As the iron added dosage increases, the nitrate removal rate of the Aspergillus sp. DH4 gradually accelerates, with a maximum dosage achieving efficient removal within 6 days. Considering the above observations, the iron added dosage of 10 g/L is selected as the optimal iron added dosage, as it achieves a relatively fast nitrate removal rate with minimal accumulation of NH₄⁺—N that could ultimately be removed through nitrification.

### Embodiment 5

Nitrogen removal performance of the Aspergillus sp. DH4 with an inorganic electron donor added at different temperatures is tested.

The Aspergillus sp. DH4 is inoculated into a sterilized DM medium with 5 mg/L of nitrogen and a C/N ratio of 2, and 10 g/L of iron is added as the inorganic electron donor; then, different temperatures of 5° C., 10° C., 15° C. and 20° C. are set as comparative experiment, and the sterilized DM medium with the Aspergillus sp. DH4 inoculated are placed in a biochemical shaker incubator at 120 rpm for culturing. Samples are taken every 2 days and filtered through pre-combusted 0.45 μm GF/F, and concentrations of NO₃⁻—N, NH₄⁺—N, NO₂⁻—N, and TN are measured under different temperatures.

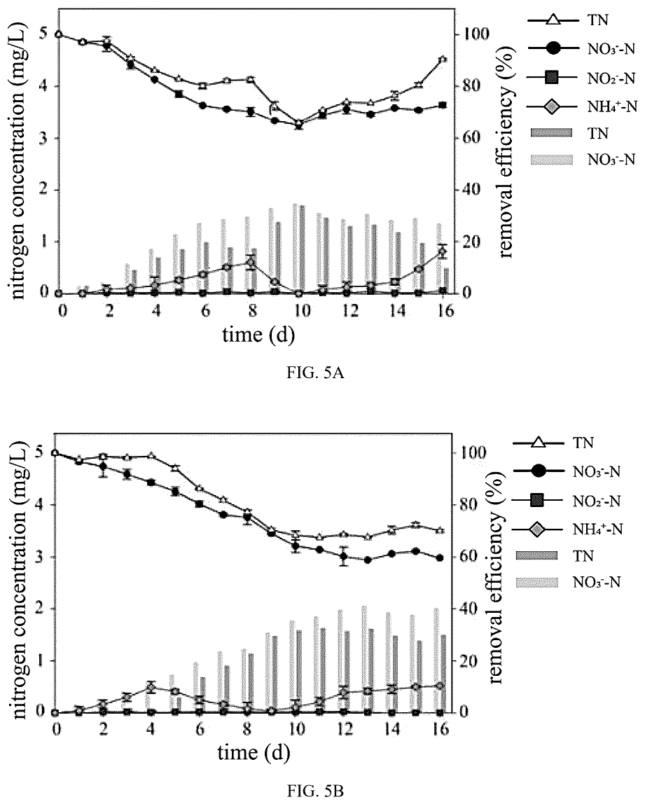

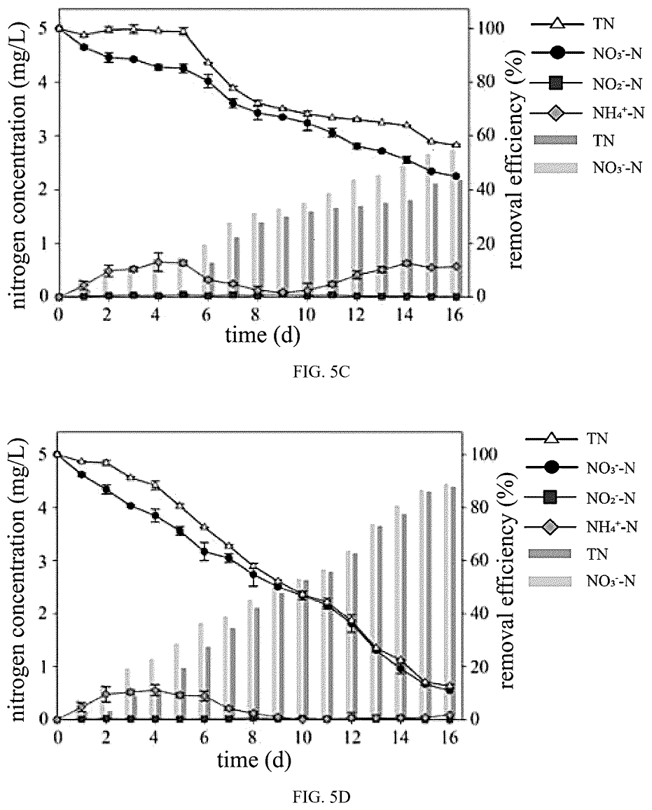

FIGS. 5A-5D show that as the temperature gradually decreases, the TN removal rate of the Aspergillus sp. DH4 decreases from 87.3% to 43.48%, 29.96%, and 9.68%. When examining the iron added dosage, at an iron added dosage of 10 g/L, a C/N ratio of 2, a rotation speed of 120 rpm, and a temperature of 30° C., the TN removal rate reaches 100%. The significant decrease in the TN removal rate compared to 30° C. indicates that the Aspergillus sp. DH4 is relatively sensitive to temperature changes. Additionally, the re-accumulation of NH₄⁺—N is observed in the later stages of cultivation under low-temperature conditions, which may be due to the release of NH₄⁺—N caused by cell death as the cells enter the decline phase, also suggesting lower cellular activity of the Aspergillus sp. DH4 at low temperatures.

Considering the overall water level and nitrogen removal situation after reservoir mixing, the disclosure selects 30° C., which brings a higher removal efficiency, as the optimal temperature condition to further investigate the nitrogen removal performance of the Aspergillus sp. DH4.

### Embodiment 6

Kinetic models for nitrogen removal performance of the Aspergillus sp. DH4 under different influencing conditions are obtained.

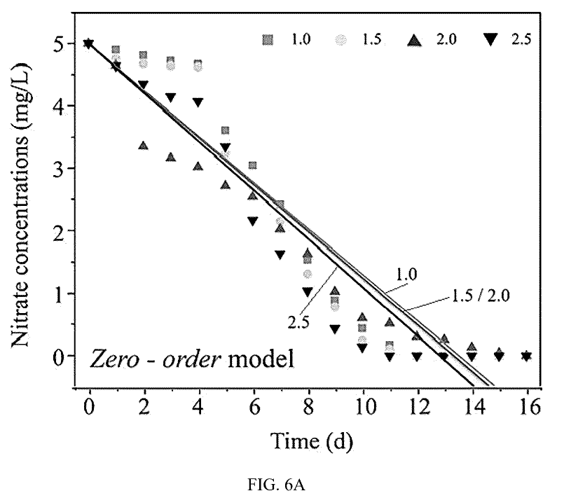

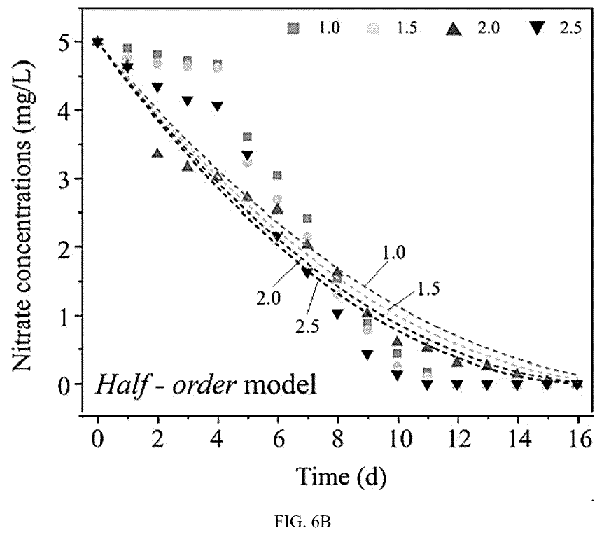

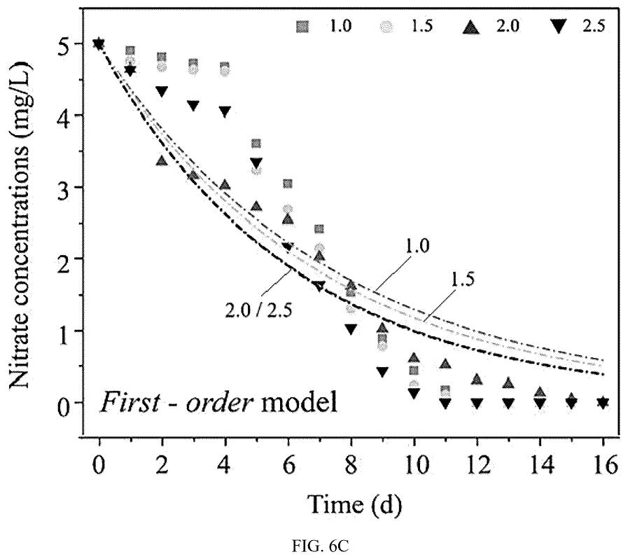

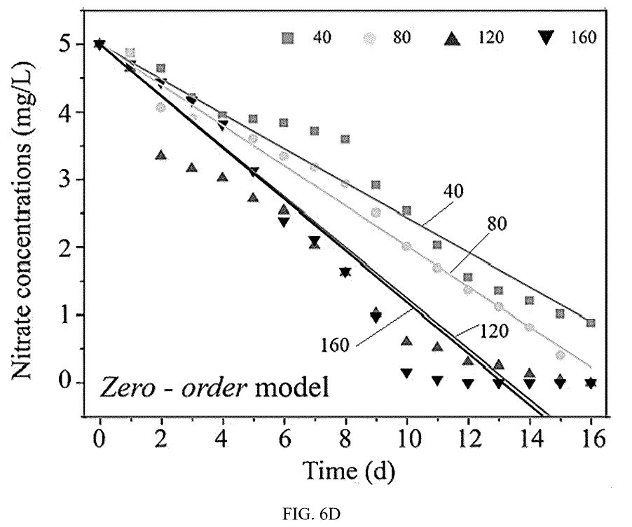

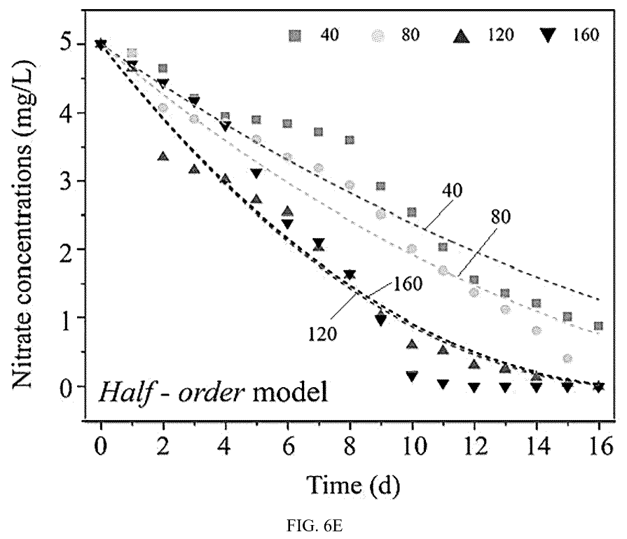

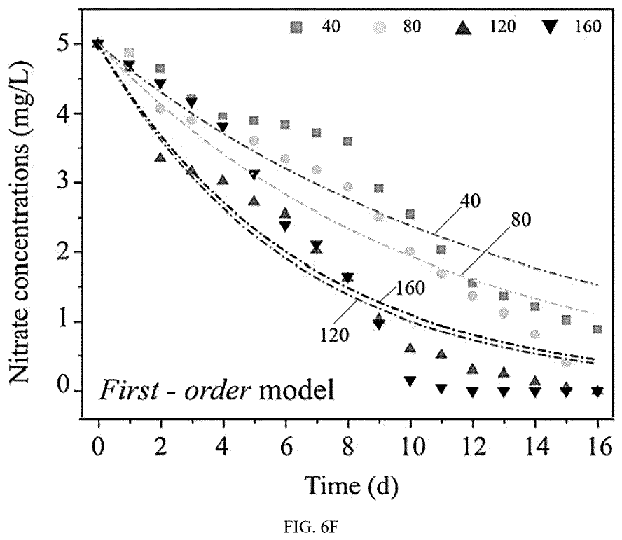

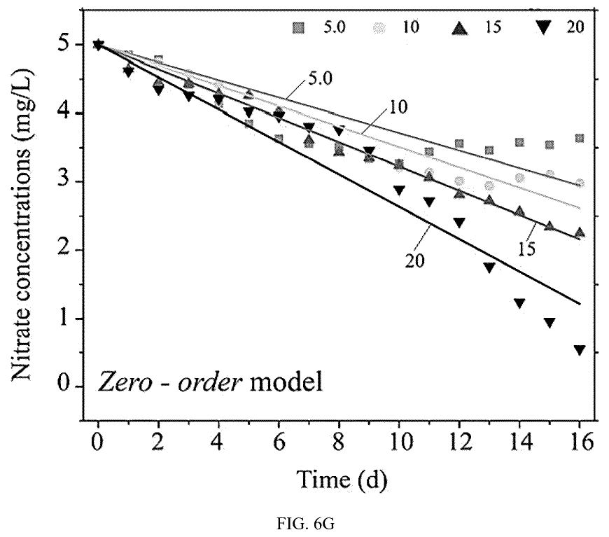

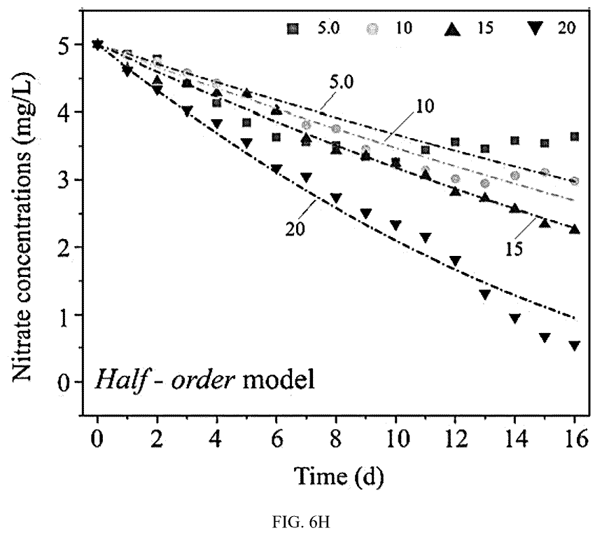

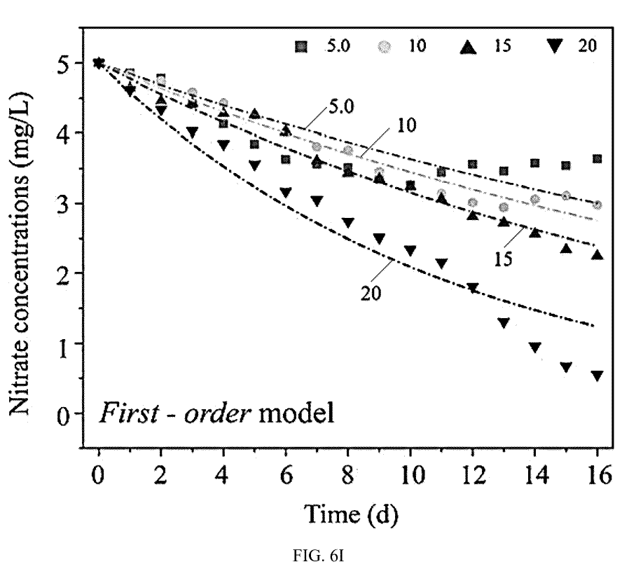

The disclosure set up changes in three different parameters, including C/N ratio, rotation speed (DO), and temperature, based on the actual seasonal variations (spring, summer, autumn, and winter) and thermal stratification phenomena in the reservoir. To specifically analyze the effects of different conditions on the nitrogen removal performance of the Aspergillus sp. DH4, the continuously measured data are fitted into different models. FIGS. 6A-6I show the zero-order, half-order, and first-order kinetic models of the Aspergillus sp. DH4 under different C/N ratios, rotation speeds (DO), and temperatures.

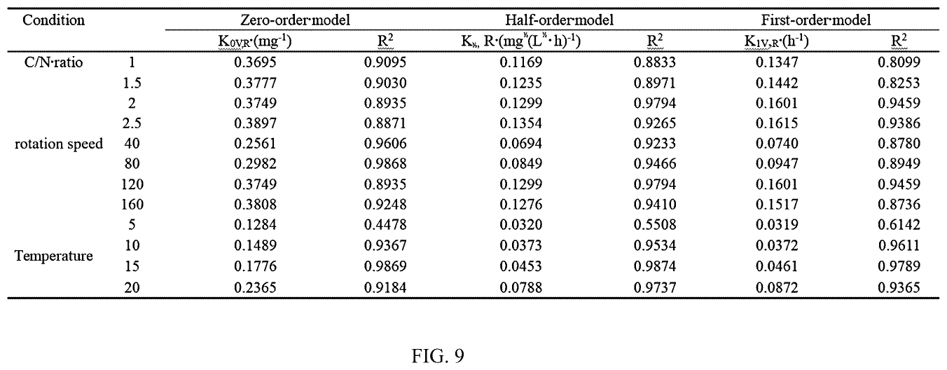

FIG. 9 illustrates the kinetic model data for nitrate removal of the Aspergillus sp. DH4 under different C/N ratios, rotation speeds (DO), and temperatures.

The zero-order, half-order, and first-order kinetic models are represented by following equations (1), (2), and (3), respectively:

$$C = {C}_{0} - {K}_{0 V , R} T \qquad ( 1 )$$

$$C = {( {C}_{0}^{1 / 2} - {K}_{1 / 2 V , R} T )}^{2} \qquad ( 2 )$$

$$C = {C}_{0} \times exp ( - {K}_{1 V , R} T ) \qquad ( 3 )$$

where C represents a nitrate concentration (mg/L) corresponding to reaction time (T, h), K_{0V,R} (mg/L) represents a reaction rate constant for the zero-order kinetic model, K_{1/2V,R} (mg^{1/2}(L^{1/2}h)⁻¹) represents a reaction rate constant for the half-order kinetic model, and K_{1V,R} (h⁻¹) represents a reaction rate constant for the first-order kinetic model.

The equation (1) represents the zero-order kinetic model, which is commonly used to indicate that the reaction rate is not limited by the concentration of the pollutant.

The equation (2) represents the half-order kinetic model, which is commonly used to indicate that environmental factors are the rate-limiting steps in pollutant removal.

The equation (3) represents the first-order kinetic model, which is commonly used to indicate that the environmental factors are the rate-limiting steps in pollutant removal, and the nitrate concentration becomes the limiting factor for the reaction rate.

FIGS. 6A-6I and FIG. 9 show that as the C/N ratio increases, R² values for the half-order kinetic model (R²=0.8833 at C/N=1.0, R²=0.9794 at C/N=2.0) and the first-order kinetic model (R²=0.8099 at C/N=0.5, R²=0.9459 at C/N=2.0) gradually increase. The maximum coefficients corresponding to the half-order and first-order kinetic models appear at a C/N ratio of 2.5, with values of 0.1354 mg^{1/2}(L^{1/2}h)⁻¹ and 0.1615 h⁻¹, respectively. As the C/N ratio increases, the denitrification rate continues to accelerate.

FIGS. 6A-6I and FIG. 9 also show that the Aspergillus sp. DH4 fits the half-order kinetic model better under varying rotation speed (rpm) conditions. As the rotation speed increases, the R² value for the half-order model gradually increases (R²=0.9233 at 40 rpm, R²=0.9794 at 120 rpm), with the maximum coefficient at 120 rpm being 0.1299 mg^{1/2}(L^{1/2}h)⁻¹. This indicates that as the rotation speed increases, the denitrification rate accelerates. However, in the half-order kinetic model, when the rotation speed increases to 160 rpm, the R² value decreases from 0.9794 to 0.9410, and the denitrification rate decreases to some extent. This suggests that excessively high DO levels can hinder mass transfer rates, thereby reducing the denitrification rate.

FIGS. 6A-6I and FIG. 9 further show that the Aspergillus sp. DH4 fits the half-order kinetic model better under varying temperature conditions. As the temperature increases, the R² value for the half-order model gradually increases (R²=0.5508 at 5° C., R²=0.9737 at 20° C.), with the maximum coefficient at 20° C. being 0.0788 mg^{1/2}(L^{1/2}h)⁻¹. This indicates that as the temperature increases, the denitrification rate accelerates.

Under varying conditions of C/N ratio, rotation speed and temperature, the denitrification rate fits the half-order rate equation better, indicating that the reaction rate is influenced by external environmental conditions. Changes in external conditions may also affect the release of Fe³⁺, thereby influencing the denitrification rate. Therefore, under natural conditions, these environmental factors do not affect the nitrate removal of the Aspergillus sp. DH4 individually but rather collectively.

### Embodiment 7

The nitrogen removal performance, and cell growth and DOC removal of the Aspergillus sp. DH4 under optimal conditions are obtained.

The Aspergillus sp. DH4 is inoculated into a sterilized DM medium with 5 mg/L of nitrogen and a C/N ratio of 2, and 10 g/L of iron is added as the inorganic electron donor; then, the sterilized DM medium are placed in a biochemical shaker incubator at 30° C. and 120 rpm for culturing. Samples are taken every 2 days and filtered through pre-combusted 0.45 μm GF/F, and concentrations of NO₃⁻—N, NO₂⁻—N, DOC and cell content are measured.

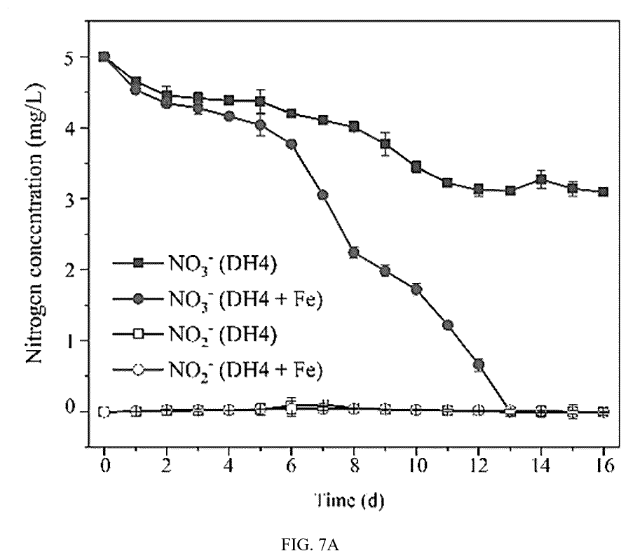

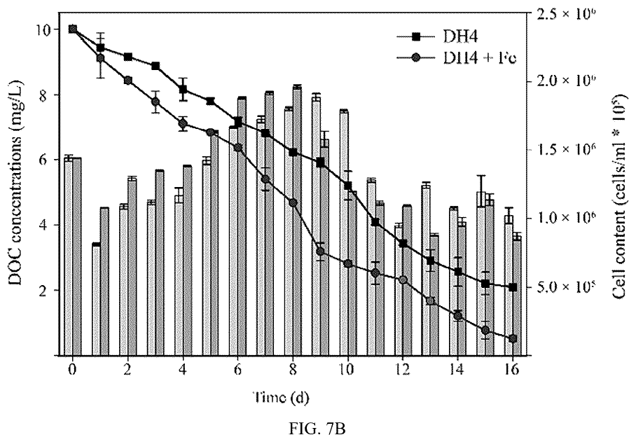

FIG. 7A and FIG. 7B show that without adding electron donors, the initial 0-1 days are an adaptation period, during which the cell count slightly decreases (from 1.54×10⁶ to 9.11×10⁵), and the nitrate removal rate is relatively slow (nitrate concentration decreases from 5 mg/L to 4.65 mg/L). During the logarithmic growth phase of the cells (5-10 days), the nitrate concentration of the Aspergillus sp. DH4 decreases from 4.37 mg/L to 3.45 mg/L, and the nitrate removal rate reaches a maximum of 37.80% on the twelfth day, followed by fluctuations.

FIG. 7A and FIG. 7B also show that when adding electron donors, the initial 0-1 days are an adaptation period, during which the cell count slightly decreased (from 1.54×10⁶ to 1.17×10⁶), and the nitrate removal rate is relatively slow (nitrate concentration decreased from 5 mg/L to 2.24 mg/L). During the logarithmic growth phase of the cells (4-8 days), the nitrate concentration of the Aspergillus sp. DH4 decreases from 4.16 mg/L to 0.00 mg/L, and the nitrate removal rate reaches 100% on the thirteenth day. Meanwhile, as nitrate is continuously removed, the DOC content also gradually decreases. This phenomenon may be due to the cells consuming DOC for self-replication while providing necessary electron donors for denitrification.

### Embodiment 8

The release of Fe³⁺ and Fe²⁺ within the system of the Aspergillus sp. DH4 is obtained.

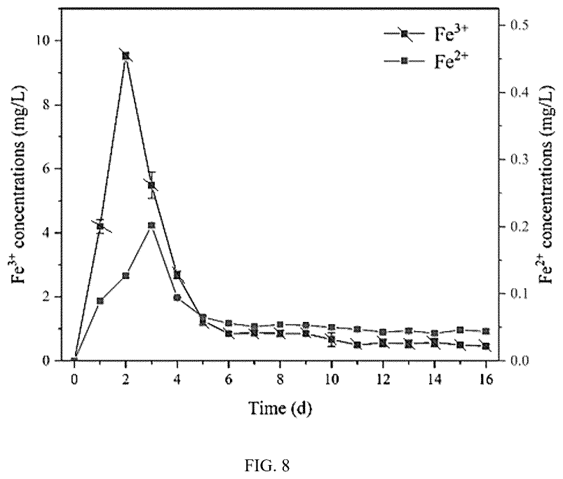

FIG. 8 shows that in the system of the Aspergillus sp. DH4, the concentrations of Fe³⁺ and Fe²⁺ first increases and then decreases. The concentration range of Fe³⁺ is relatively large, reaching a maximum of 1.63 mg/L on the second day, after which it rapidly decreases and fluctuates around 0.60 mg/L. This indicates that the oxidation speed of the iron initially is fast but gradually slows down, with the formation of iron oxides on the surface blocking subsequent iron oxidation, although it continued at a slower pace, leading to the observed fluctuations in concentration. The concentration of Fe²⁺ is relatively low, peaking at 0.20 mg/L on the third day, and then fluctuating within the range of 0.14-0.054 mg/L. The likely reason is that under aerobic conditions, the rate of conversing Fe²⁺ to Fe³⁺ is relatively fast, resulting in a lower concentration of Fe²⁺.

It should be noted that when the claims of the disclosure involve numerical ranges, it should be understood that the two endpoints of each numerical range and any value between the two endpoints can be selected. To avoid repetition, the disclosure describes preferred embodiments.

Although illustrated embodiments of the disclosure have been described, those skilled in the art may make additional changes and amendments to these illustrated embodiments once they have knowledge of the basic inventive concept. Therefore, the appended claims are intended to be interpreted as including the illustrated embodiments and all changes and amendments thereto falling within the scope of protection of the disclosure.

Apparently, those skilled in the art can make various modifications and variations to the disclosure without departing from the spirit and scope of the disclosure. Thus, if these modifications and variations of the disclosure fall within the scope of protection of the appended claims and their equivalent solutions, the disclosure is also intended to include these modifications and variations therein.

What is claimed is:

1. A use of an Aspergillus sp. DH4 with aerobic denitrification enhanced by an inorganic electron donor, comprising:
applying the Aspergillus sp. DH4 in restoring a nitrogen-containing water body;
wherein the Aspergillus sp. DH4 is preserved at China Center for Type Culture Collection (CCTCC), a preservation number is CCTCC NO: M20232690, and a preservation date is Dec. 27, 2023;
wherein the inorganic electron donor is zero-valent iron; and
wherein the restoring a nitrogen-containing water body comprises: removing nitrogen from the nitrogen-containing water body.

2. The use as claimed in claim 1, wherein a carbon to nitrogen ratio of the nitrogen-containing water body is in a range of 1-2.5, and a temperature of the nitrogen-containing water body is in a range of 25-30 degrees Celsius (° C.).

3. The use as claimed in claim 1, wherein during the restoring a nitrogen-containing water body, a usage amount of the Aspergillus sp. DH4 is 1%-5% of the nitrogen-containing water body in percentages by weight.

4. The use as claimed in claim 1, wherein an added dosage of the zero-valent iron is in a range of 10-15 grams per liter (g/L).

5. The use as claimed in claim 4, wherein the zero-valent iron is a zero-valent iron powder or a zero-valent iron rod.

6. A method for removing nitrate from a polluted water body, comprising:
inoculating the Aspergillus sp. DH4 with aerobic denitrification enhanced by the inorganic electron donor as claimed in claim 1 into the nitrogen-containing water body, wherein the zero-valent iron is added as the inorganic electron donor.


SEQUENCE LISTING (1 sequence)

SEQ ID NO: 1 - DNA, 533 bp, synthetic construct
  source 1..533: mol_type = other DNA
ctgcggaaggatcattaccgagtgcgggctgcctccgggcgcccaacctcccacccgtgactacctaacactgttgcttcggcggggagccccctaggggcgagccgccggggaccactgaacttcatgcctgagagtgatgcagtctgagcctgaatacaaatcagtcaaaactttcaacaatggatctcttggttccggcatcgatgaagaacgcagcgaactgcgataagtaatgtgaattgcagaattcagtgaatcatcgagtctttgaacgcacattgcgccccctggcattccggggggcatgcctgtccgagcgtcattgctgccctcaagcccggcttgtgtgttgggtcgtcgtcccccccgggggacgggcccgaaaggcagcggcggcaccgtgtccggtcctcgagcgtatggggctttgtcacccgctcgattagggccggccgggcgccagccggcgtctccaaccttatttttctcaggttgacctcggatcaggtagggatacccgctgaacttaa